(19) United States
(12) Patent Application Publication
(10) Pub. No.: US 2018/0280161 A1
(43) Pub. Date: Oct. 4, 2018

(54) BREAST PROSTHESIS

(71) Applicant: Karen C. Roper, Bloomington, IN (US)

(72) Inventor: Karen C. Roper, Bloomington, IN (US)

(21) Appl. No.: 15/825,416

(22) Filed: Nov. 29, 2017

Related U.S. Application Data
(60) Provisional application No. 62/478,888, filed on Mar. 30, 2017.

(51) Int. Cl. A61F 2/52; A61F 2/78

(52) U.S. Cl. CPC A61F 2/52; A61F 2002/7868; A61F 2/78

(57) ABSTRACT

A breast prosthesis is provided for women who have undergone a double (or single) mastectomy. The prosthesis comprises a breast form that substantially resembles a human breast and a back panel to which the breast form can be coupled. The breast form has at least one magnet adjacent a wearer's chest when the prosthesis is worn. The back panel also has at least one magnet that faces outwardly from the chest when the prosthesis is worn. When a wearer wishes to don the prosthesis, she places the back panel against her chest in a desired location, with the magnets facing outwardly. A garment may then be placed over the panel before the breast form is placed over the garment. The back panel and the breast form may then be coupled to one another via their magnets (which attract one another) so that the prosthesis is secured in a desired position.

### CROSS-REFERENCE TO RELATED APPLICATIONS

This application claims the benefit of U.S. Provisional Application No. 62/478,888, filed Mar. 30, 2017, which is hereby incorporated by reference in its entirety.

### FIELD OF THE INVENTION

The present invention relates to a breast prosthesis that may be worn by women who have undergone double (or single) mastectomies. More specifically, the invention is a two-part breast prosthesis which may be magnetically coupled to a garment as an alternative to other breast prostheses available on the market.

### BACKGROUND OF THE INVENTION

The number of women choosing to have a breast reconstruction after a mastectomy has been steadily increasing. However, breast reconstruction involves substantial recovery time, and it also comes with health risks associated with major surgery. Thus, some women prefer not to have reconstruction surgery and decide instead to move forward by wearing various types of breast prostheses. Such wearable prosthetic breast forms come in many materials and garment-types. Breast forms can be made from materials such as silicone gel, foam, or fiberfill, to create a similar weight, feel, and shape to a natural breast.

The most simple of prosthetic breast forms use an adhesive on the back side of the breast form to adhere the breast form directly to the chest area. However, a tight adhesive fit to the chest may trap heat and prevent ventilation of the skin surface. This may be uncomfortable and further may cause skin irritation.

Other breast form solutions fit into special bra pockets that help hold the prosthesis in place. However, bras are uncomfortable, and many women would prefer not to have to wear them if they don't have to. Also, for women who have recently undergone a mastectomy, lymphedema may develop at the surgery site. Restrictive, tight fitting bras may cause a great deal of pain in these sites where surgery took place and lymphedema is present.

Even moreover, a prosthetic breast form in a bra pocket may shift out of position when in use, and depending on the blouse or shirt worn by a woman, a different type of bra for prostheses may be needed. This can generate substantial expense, and such expense may not be covered by medical insurance.

In U.S. Pat. No. 6,074,420 to Eaton, 7,058,439 to Eaton, and U.S. application 2015/0128962 to Sinnecker, magnets may be used to attach the breast prosthesis to a woman's chest. However, that prosthesis involves implanting magnets underneath the skin that may be selectively coupled to magnets contained within a breast form. This solution can be problematic, however, because the magnets implanted below the skin surface may cause discomfort or skin irritation. It also may present issues when a user must undergo a diagnostic procedure like an MRI scan, which cannot be conducted with magnets present in the body.

### SUMMARY OF THE INVENTION

A breast prosthesis that does not require the use of a bra, and does not require a procedure to implant any foreign objects below the skin, is disclosed herein. The breast prosthesis may generally comprise each of a breast form portion and a back panel portion which are selectively coupled to one another. The breast form is shaped substantially like a human breast, while the back panel substantially conforms to the shape of the chest wall. A camisole or other non-restrictive garment that does not require underwiring like bras may be placed between the breast form and back panel when the prosthesis is worn to help secure the prosthesis in place.

Both the breast form and back panel have an anterior and posterior portion. The breast form has at least one magnet or ferromagnetic member on its posterior portion, and the back panel also has at least one magnet or ferromagnetic member on its anterior portion. The at least one magnet or other ferromagnetic member of the breast form and back panel are preferably attracted to one another and allow the breast form and back panel to magnetically couple to one another.

To don the prosthesis, the back panel should first be placed on the wearer's chest in the location where the wearer would like the breast form to sit. The back panel should be placed underneath a garment (e.g., a camisole). The breast form may then be placed on top of the garment. The breast form and back panel may then be arranged so that the at least one magnet or ferromagnetic member of the breast form and back panel may couple and magnetically attach to one another. After this takes place, the garment is secured between the breast form and the back panel.

When the wearer wishes to remove the breast prosthesis, she may pull the breast form away from the back panel with sufficient force to disengage its magnet or ferromagnetic member from the magnet or ferromagnetic member with which it was coupled on the back panel. Then, the back panel may simply be removed from being adjacent the wearer's chest, and the wearer is left only wearing her undergarment shirt.

### BRIEF DESCRIPTION OF THE DRAWINGS

For a better understanding of the various embodiments of the present invention, reference may be made to the accompanying drawings in which:

FIG. 1 is an exploded perspective front view of the breast prosthesis and garment;

FIG. 2 is an exploded perspective rear view of the breast prosthesis and garment of FIG. 1;

FIG. 3 is a perspective rear view of the breast form of FIGS. 1 and 2;

FIG. 4 is an exploded perspective rear view of the breast form of FIGS. 1-3;

FIG. 5 is a cross-section of the breast form taken across line 5-5 of FIG. 10;

FIG. 6 is an exploded perspective view of the pocket members of FIGS. 1, 2 and 4;

FIG. 7 is a perspective rear view of the back panel of FIGS. 1 and 2;

FIG. 8 is an exploded perspective rear view of the back panel of FIGS. 1, 2 and 7;

FIG. 9 is a cross-section of the back panel taken across line 9-9 of FIG. 10; and

FIG. 10 is a left elevation view of the breast prosthesis and garment of FIGS. 1 and 2 in its operative position.

While the disclosure is susceptible to various modifications and alternative forms, specific embodiments thereof are shown by way of example in the drawings and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure.

### DETAILED DESCRIPTION OF THE DRAWINGS

The invention will now be described with reference to the drawing figures, in which like reference numerals refer to like parts throughout. For purposes of clarity in illustrating the characteristics of the present invention, proportional relationships of the elements have not necessarily been maintained in the drawing figures.

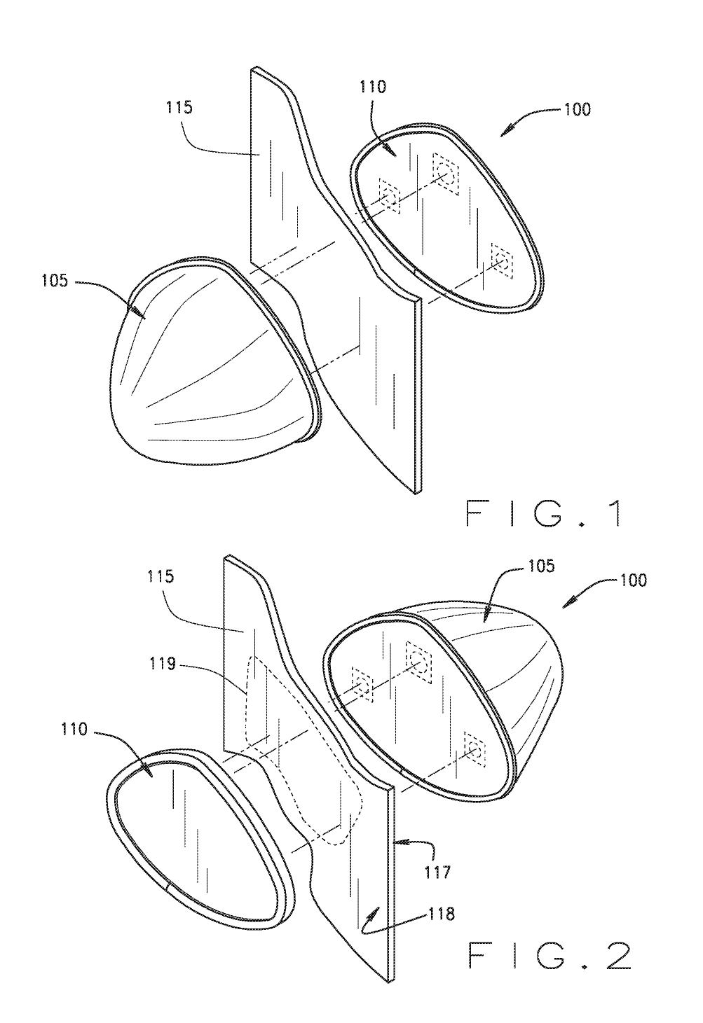

A breast prosthesis 100 is provided in FIGS. 1 and 2 that when worn, substantially resembles the look and feel of a natural human breast. Two breast prostheses 100 may be worn by women who have had a double mastectomy, or a single breast prosthesis 100 may be worn in the event of a single mastectomy. The breast prosthesis 100 generally comprises a breast form 105 and a back panel 110, which may be coupled to a garment 115 (as described in more detail below).

Breast form 105 preferably has a shape that substantially resembles a human breast. Breast form 105 may resemble the natural shape of a breast or the shape of a breast while wearing a bra. As such, the breast form 105 may be comprised of a variety of materials that mimic the shape and feel of a breast. The back panel 110, on the other hand, has a shape that substantially conforms to the chest wall of the person using the prosthesis. The back panel 110 is preferably made of one or more fabrics that do not cause discomfort when placed against the skin of a wearer. The size of the breast form 105 and back panel 110 may differ according to the preferences of the wearer.

Garment 115 may be a variety of garment types, and includes an anterior side 117 and a posterior side 118. The anterior side 117 of the garment 115 faces outwardly from a wearer and is visible when the garment 115 is worn. The posterior side 118 of garment 115 is preferably the side adjacent to the wearer and cannot be seen when garment 115 is worn.

In one embodiment, either or both the anterior side 117 or posterior side 118 may include at least one pocket 119 shaped for receiving the back panel 110 or breast form 105. Pocket 119 may further reduce movement of the breast form 105 or back panel 110 when breast form 105 or back panel 110 is secured within pocket 119.

In at least one other embodiment, the garment 115 may be a non-restrictive, but securely fitting camisole that may be worn under a variety of garments as an undershirt. In another embodiment, garment 115 may be any top, or garment worn on the upper body, where at least the front half of the top has a fitted layer, including where a front half of a fitted top is sewn onto and underneath another top.

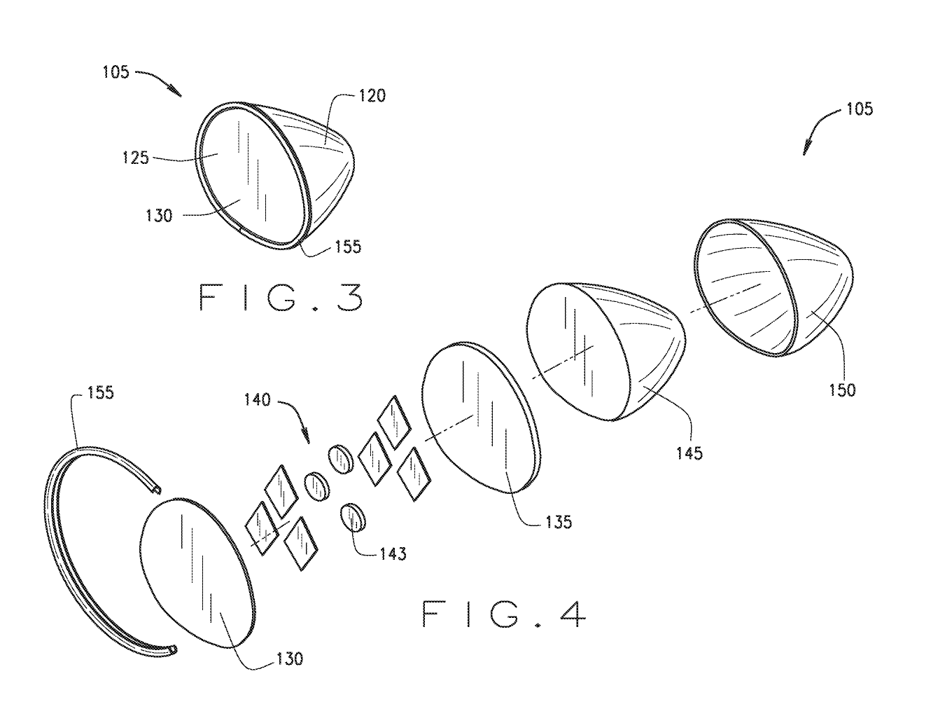

Turning to FIGS. 3 and 4, the breast form 105 is illustrated in greater detail. As illustrated, the breast form 105 includes an anterior portion 120 and a posterior portion 125. For reference, the anterior portion 120 is the portion of the breast form 105 that projects away from a wearer, while the posterior portion 125 is the portion adjacent to the garment 115 when the prosthesis 100 is worn.

In one embodiment, the breast form 105 includes a first polyester layer 130, preferably positioned adjacent to the anterior side 117 of garment 115 when the breast prosthesis 100 is worn, and covers the posterior portion 125 of the breast form 105. Breast form 105 further preferably includes a first semi-rigid layer 135 which provides a surface for at least one pocket member 140 to be attached to, so that at least one pocket member 140 may be provided between the first polyester layer 130 and the first semi-rigid layer 135 when constructed. Thus, at least one pocket member 140, which may serve as a receptacle for a magnet 143 (described in more detail hereinafter), may be sewn or otherwise attached onto the first semi-rigid layer 135 of the breast form 105. In alternative embodiments, at least one pocket member 140 may be attached to the first polyester layer 130, or attached to both the first polyester layer 130 and the first semi-rigid layer 135.

In the illustrated embodiment of FIG. 4, breast form 105 includes three pocket members 140, which each contains a magnet 143. The first semi-rigid layer 135 is located and positioned adjacent and between both the first polyester layer 130 (or at least one pocket member 140) and the breast shaped memory foam 145.

A spandex layer 150 is preferably provided that is substantially in the shape of the breast shaped memory foam 145. The spandex layer 150 preferably receives and secures breast shaped memory foam 145. The visible outer layers of breast form 105 may include spandex layer 150 and first polyester layer 130, wherein spandex layer 150 receives the breast shaped memory foam 145, first semi-rigid layer 135, and pocket members 140.

A first bias tape 155 covers the raw edge or perimeter of both the spandex layer 150 and the first polyester layer 130 by enveloping the perimeters of the spandex layer 150 and the first polyester layer 130. The first bias tape 155 can be secured around the perimeter of both the spandex layer 150 and the first polyester layer 130 by sewing, gluing, or any other method known in the art, thereby preferably preventing the perimeters of the spandex layer 150 and first polyester layer 130 from causing discomfort to the wearer.

As shown in FIGS. 4 and 5, the posterior portion 125 may include three magnets 143 substantially arranged in the shape of an equilateral triangle. In other embodiments, there may be more or fewer magnets 143. Alternative arrangements of the magnets 143 are also envisioned and foreseeable. The magnets 143 are preferably magnets that are strong enough to securely couple to ferromagnetic members (discussed below) of the back panel 110, even when material such as the garment 115 is placed between the breast form 105 and the back panel 110. Yet, the magnets 143 also should not require an exceedingly high degree of strength to be decoupled from one another. The magnets 143 thus may be permanent samarium-cobalt or neodymium-iron-boron magnets, though other magnet types are also envisioned herein.

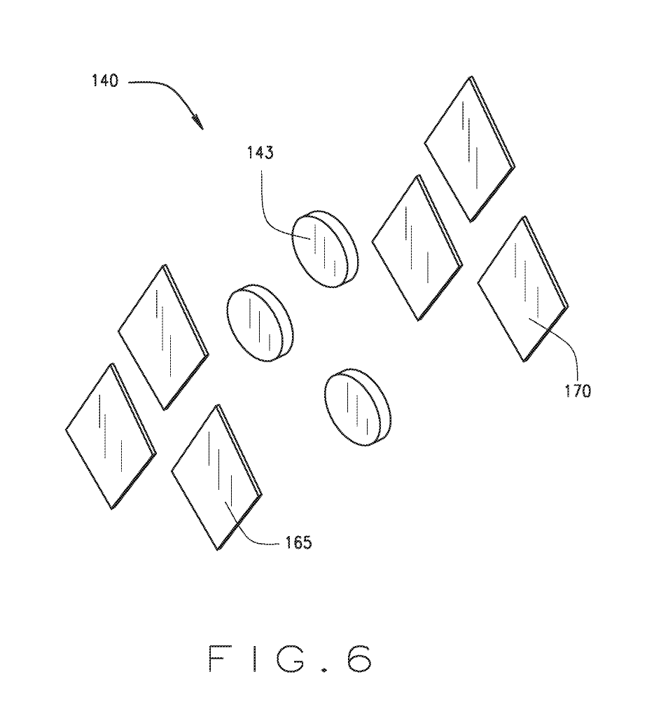

The magnets 143 may be secured at a certain location within the posterior portion 125 of the breast form 105 by pocket members 140. As shown in FIG. 6, pocket members 140 may serve as receptacles for magnets 143, wherein the magnet 143 is preferably placed and secured between a first semi-rigid rectangular fabric 165 and a second semi-rigid rectangular fabric 170. In greater detail, the pocket members 140 may be formed by placing magnets 143 between the first semi-rigid rectangular fabric 165 and the second semi-rigid rectangular fabric 170, and stitching around the magnets 143 in a manner known and understood in the art to secure the magnets 143 in a specific location within a receptacle. Magnets 143 can also be secured between the first semi-rigid rectangular fabric 165 and the second semi-rigid rectangular fabric 170 by glue, snaps, or any other method known in the art or foreseeable. The pocket members 140 themselves may be secured by sewing, gluing, buttons, snaps, or some other combination thereof to the first semi-rigid layer 135 of the breast form 105 and/or the first polyester layer 130.

In an alternative embodiment, pocket members 140 can be formed without enclosing the magnets 143 within a receptacle. Instead, pocket members 140 are formed by placing the magnets 143 between the first semi-rigid layer 135 and the first polyester layer 130 of breast form 105. Stitches may be sewn through the first semi-rigid layer 135 and the first polyester layer 130 and around the magnet 143 to hold it in place. Pocket members 140 may be formed by any method known in the art that secures magnets 143 to the breast form 105.

Substantially similar to breast form 105, the back panel 110 includes an anterior portion 172 and a posterior portion 174 (see FIG. 7). The anterior portion 172 of the back panel 110 preferably faces outwardly, adjacent to posterior side 118 of garment 115 when the breast prosthesis 100 is worn. The posterior portion 174, on the other hand, is adjacent to the wearer's chest when the breast prosthesis 100 is worn. Both the anterior portion 172 and the posterior portion 174 are preferably flat but semi-rigid so that they may take on the shape of the wearer's chest cavity when worn, but also reduces the possibility of the magnets 143 and/or ferromagnetic members 163 of back panel 110 coupling to each other.

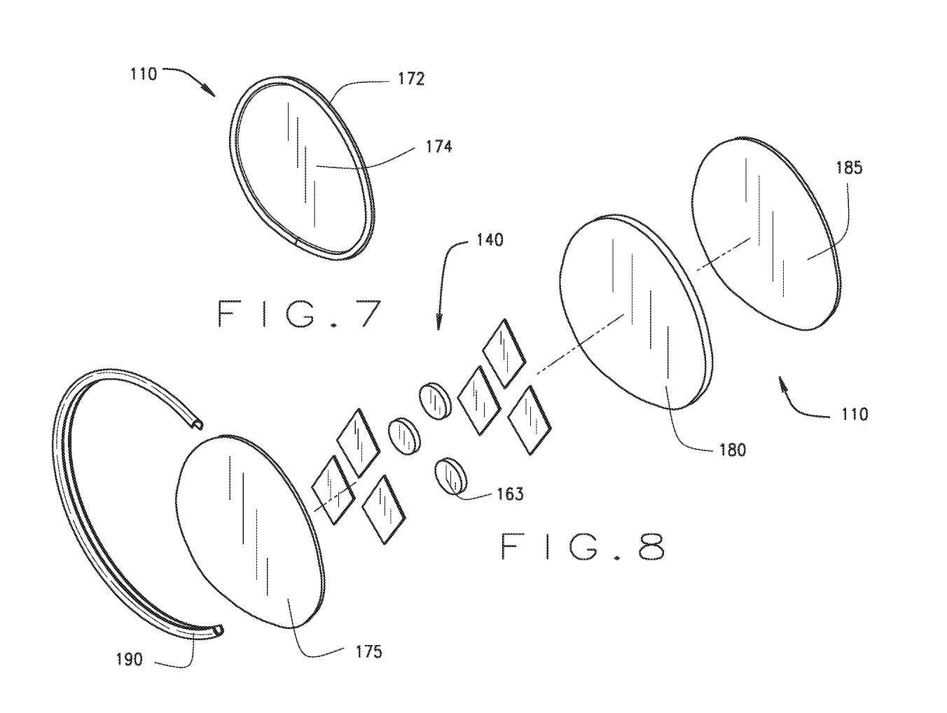

Turning to FIG. 8, the back panel 110 further includes a second polyester layer 175, located and positioned adjacent to the posterior side 118 of the garment 115, covering the anterior portion 172 of the back panel 110. The second polyester layer 175 may also be adjacent to a second semi-rigid layer 180, thereby allowing the back panel 110 to conform to a wearer's chest cavity when worn and reduce the possibility that magnets 140 or ferromagnetic members 163 on the back panel 110 will couple to one another. At least one pocket member 140 may be located and positioned between the second polyester layer 175 and the second semi-rigid layer 180, and may be attached to either or both layers by sewing, gluing, or other methods known in the art, substantially similar to at least one pocket member 140 in the breast form 105. The posterior portion 174 of the back panel 110 is preferably covered by a jersey layer 185, located and positioned adjacent to the wearer's chest. The perimeter or edges of the back panel 110, or more specifically, the perimeters of the second polyester layer 175, second semi-rigid layer 180, and jersey layer 185 are enveloped by the second bias tape 190, thereby preferably preventing discomfort to the wearer. The second bias tape 190 may be secured by sewing, gluing, or any other method known in the art, substantially similar to first bias tape 155.

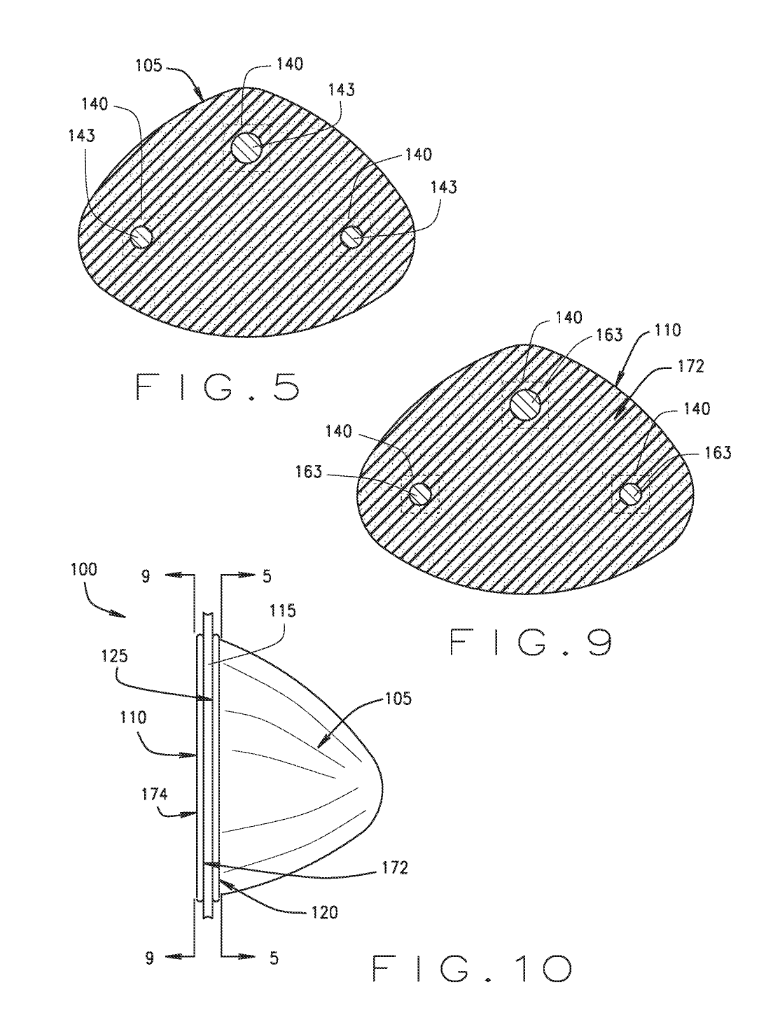

In one embodiment, the pocket members 140 of the back panel 110 preferably has at least one magnet 143 or ferromagnetic member 163 embedded therein. In the cross-section of FIG. 9, the anterior face 172 includes three ferromagnetic members 163 substantially arranged as an equilateral triangle, similar to the magnets 143 of the breast form 105. In alternative embodiments, ferromagnetic members 163 may be replaced by magnets 143 and vice versa, as long as the magnets within the breast form 105 and back panel are still able to attract and able to couple to one another. In yet another alternative embodiment, the magnets 143 and/or ferromagnetic members 163 are not embedded within the breast form 105 or back panel 110, respectively. Instead, the magnets 143 and/or ferromagnetic members 163 may be exposed. Alternatively, they may be covered and/or secured using a mechanism separate from the pocket members 140. Other methods in which the magnets 143 or ferromagnetic members 163 are attached to the breast form 105 or back panel 110 are also envisioned and foreseeable. The magnets 143 or ferromagnetic members 163 of the breast form 105 and back panel 110 increase the weight of the breast form 105 and back panel 110. Therefore, breast form 105 and back panel 110 may be weighted and may remain firmly in place regardless of the activity of the wearer. Foam prostheses without magnets are a light-weight alternative, but they will often shift extensively when exercising or swimming. Silicon breast prostheses, on the other hand, may be weighted. Thus, they may remain in place when exercising or swimming, but may react adversely with chlorinated water. Therefore, breast prosthesis 100 may be a good alternative for women who need or desire a light-weight alternative that would allow them to exercise or swim without worrying about extensive movement of the breast prosthesis or adverse reactions.

To assemble the breast prosthesis 100 for wear (as shown in FIG. 10), the wearer first places the back panel 110 so that its posterior portion 174 is facing the chest wall in a location on the chest where the wearer prefers that the breast prosthesis 100 be located. The garment 115 may then be placed over the anterior portion 172 of the back panel 110. Subsequently, the posterior face 125 of the breast form 105 may be placed against the anterior side 117 of the garment 115.

The magnets 143 or ferromagnetic members 163, associated with the back panel 110, may be secured in a specific location by pocket members 140 of the back panel 110 that are in substantial alignment with the pocket members 140 of the breast form 105. Thus, when a wearer is ready to attach the back panel 110 to the breast form 105, the ferromagnetic members 163 of the back panel 110 and the magnets 143 of the breast form 105 are already aligned, and the wearer does not need to fumble with either the back panel 110 or breast form 105 while trying to align the back panel 110 and breast form 105 in a desired location.

When the magnets 143 and ferromagnetic members 163 of the breast form 105 and back panel 110 are aligned, they will magnetically couple with the garment 115 and secure the garment 115 between the breast form 105 and the back panel 110. Both the breast form 105 and back panel 110 may have magnets 143, ferromagnetic members 163, or a combination of such, as long as the magnet 143 and/or ferromagnetic member 163 in the breast form 105 is attracted to its corresponding magnet 143 and/or ferromagnetic member 163 in the back panel 110.

Thus, when worn, the posterior portion 125 of the breast form 105 is coupled with the anterior portion 172 of the back panel 110, so that the magnets 143 and ferromagnetic members 163 are magnetically attached to one another. The breast form 105 and the back panel 110 are preferably coupled in a manner that secures the garment 115 there between. The garment 115 allows the wearer to feel as though she is wearing an undergarment, and it also allows for some movement of the breast prosthesis 100 when the garment 115 moves. This allows for some movement of the breast prosthesis 100 that resembles the natural movement of a breast when it is contained in an undergarment like a bra.

The garment 115 may be any fitted upper body garment or top. In one embodiment, garment 115 is preferably formed out of an elastic material, such as Spandex, so that it clings to the body of the wearer. In an alternative embodiment, garment 115 may be a fitted camisole. In yet another embodiment, garment 115 may be any upper body garment where at least the front half of the garment has a fitted layer. This may include a blouse or other garment sewn onto and underneath another garment. The garment 115 may also include shoulder straps so that the weight of the breast prosthesis 100 may be transferred to the shoulders. The garment 115 may be provided by the wearer or be provided along with the breast prosthesis 100. If garment 115 is supplied with breast prosthesis 100, then the material that they are formed out of may be the same to allow the breast prosthesis 100 and garment 115 to blend together.

In alternative embodiments, the garment 115 may also include pockets 119 in which the breast form 105 or back panel 110 may be secured, as discussed hereinabove and shown in FIG. 2. In this embodiment, the pockets 119 would serve to further secure the breast form 105 in place (in conjunction with the magnets 143 or ferromagnetic members 163). In a separate alternative embodiment, the garment 115 may be modified so that in the case of a single mastectomy, a pocket is provided in a modified garment (or bra) for a single breast form 105. The modified bra would be altered so that it only includes a single cup, whereas the wearer could wear a single cup bra and the prosthesis 100 at the same time. Alternatively, the wearer could wear a normal bra and place the prosthesis 100 within a cup. In all the alternative embodiments described above, the magnets 143 or ferromagnetic members 163 may be provided in the breast form 105 as described above. The magnets 143 or ferromagnetic members 163 may be provided in a back panel 110 substantially similar to breast form 105. Alternatively, magnets 143 or ferromagnetic members 163 may be provided in the garment 115 itself, thereby eliminating the need for back panel 110. Pocket members 140 or other means would directly attach magnets 143 or ferromagnetic members 163 to garment 115.

In describing the breast prosthesis 100 above, the prosthesis 100 has been described as a single apparatus, comprised of the breast form 105, the back panel 110, and the garment 115. However, in the case of a double mastectomy, it should be understood that two such prosthesis 100 would be used together to resemble two breasts.

From the foregoing, it will be seen that the various embodiments of the present invention are well adapted to attain all the objectives and advantages hereinabove set forth together with still other advantages which are obvious and which are inherent to the present structures. It will be understood that certain features and sub-combinations of the present embodiments are of utility and may be employed without reference to other features and sub-combinations. Since many possible embodiments of the present invention may be made without departing from the spirit and scope of the present invention, it is also to be understood that all disclosures herein set forth or illustrated in the accompanying drawings are to be interpreted as illustrative only and not limiting. The various constructions described above and illustrated in the drawings are presented by way of example only and are not intended to limit the concepts, principles and scope of the present invention.

Many changes, modifications, variations and other uses and applications of the present invention will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A breast prosthesis comprising:
a breast form having a shape that substantially resembles a human breast;
the breast form having at least one magnet; and
a back panel having a shape that substantially conforms to a chest wall;
the back panel having at least one magnet attracted to the at least one magnet of the breast form.

2. The breast prosthesis of claim 1 wherein the breast prosthesis includes a garment positioned and located between the breast form and the back panel, wherein the garment is secured between the breast form and the back panel when the at least one magnet of the breast form and the back panel are magnetically couple to one another.

3. The breast prosthesis of claim 1 wherein the at least one magnet of the back panel is located in an anterior portion of the back panel.

4. The breast prosthesis of claim 1 wherein the at least one magnet of the breast form is located in a posterior portion of the breast form.

5. The breast prosthesis of claim 1 wherein the breast form and the back panel have at least three magnets.

6. The breast prosthesis of claim 1 wherein the breast form is made out of memory foam.

7. The breast prosthesis of claim 1 wherein the at least one magnet of the breast form and the at least one magnet of the back panel are samarium-cobalt.

8. The breast prosthesis of claim 1 wherein the back panel includes a polyester layer and a jersey layer.

9. The breast prosthesis of claim 1 wherein the breast form and the back panel each include at least one pocket member for securing the at least one magnet of the breast form and the at least one magnet of the back panel in place.

10. The breast prosthesis of claim 9 wherein the at least one pocket member of the breast form and the pocket member of the back panel are each stitched in the breast form and the back panel, respectively.

11. The breast prosthesis of claim 1 wherein the breast form and the back panel each include a flap for securing the at least one magnet of the breast form and the at least one magnet of the back panel in place.

12. The breast prosthesis of claim 1 wherein the breast form has a shape of a breast when wearing a bra.

13. A breast prosthesis comprising:
a breast form having a shape that substantially resembles a human breast, the breast form having at least one magnet;
a back panel having a shape that substantially conforms to a chest wall, the back panel having at least one magnet;
a garment positioned and located between the breast form and the back panel so that when the at least one magnet of the breast form and the at least one magnet of the back panel are magnetically coupled, the garment is secured between the two at least one magnets.

14. The breast prosthesis of claim 13 wherein the garment is a camisole.

15. The breast prosthesis of claim 13 wherein the breast form and the back panel each include at least one pocket member for securing the at least one magnet of the breast form and the at least one magnet of the back panel in place.

16. The breast prosthesis of claim 13 wherein the at least one magnet of the back panel is located in an anterior portion of the back panel.

17. The breast prosthesis of claim 13 wherein the at least one magnet of the breast form is located in a posterior portion of the breast form.

18. The breast prosthesis of claim 13 wherein the breast form is made out of memory foam.

19. The breast prosthesis of claim 13 wherein the at least one magnet of the breast form is neodymium-iron-boron.

20. The breast prosthesis of claim 13 wherein there are at least two magnets on each of the breast form and the back panel.
